(19) United States
(12) Patent Application Publication
(10) Pub. No.: US 2006/0030863 A1
(43) Pub. Date: Feb. 9, 2006

(54) Implanted bronchial isolation device delivery devices and methods

(76) Inventors: Antony J. Fields, San Francisco, CA (US); John McCutcheon, Menlo Park, CA (US); Alan R. Rapacki, Redwood City, CA (US)

Correspondence Address: FISH & RICHARDSON, PC, P.O. BOX 1022, MINNEAPOLIS, MN, 55440-1022, US

(21) Appl. No.: 11/186,293

(22) Filed: Jul. 20, 2005

Related U.S. Application Data
(60) Provisional application No. 60/590,167, filed on Jul. 21, 2004.

(51) Int. Cl. A61F 11/00

(52) U.S. Cl. 606/108; 600/129

(57) ABSTRACT

Disclosed is a delivery system for insertion into a lung. The delivery system includes an elongate endoscope having a proximal end and a distal end and defining an internal lumen. The endoscope is configured to articulate in one or more directions. The system further includes a catheter that includes an elongate shaft sized to be inserted through the internal lumen of the endoscope. The catheter has a shape that aids in the articulation of the distal end of the endoscope when the catheter is positioned inside the endoscope.

### REFERENCE TO PRIORITY DOCUMENT

This application claims priority of co-pending U.S. Provisional Patent Application Ser. No. 60/590,167 entitled “Implanted Bronchial Isolation Device Delivery Devices and Methods”, filed Jul. 21, 2004. Priority of the aforementioned filing date is hereby claimed, and the disclosure of the Provisional Patent Application is hereby incorporated by reference in its entirety.

### BACKGROUND

This disclosure relates generally to methods and devices for use in performing pulmonary procedures and, more particularly, to delivery devices and procedures for the lungs.

Lung interventional procedures often require that one or more catheters be placed into various locations in the lungs. The types of lung interventional procedures that require the use of catheters can vary. Some exemplary types of procedures include lung region isolation procedures, temporary bronchial occlusion procedures, and drug delivery, lavage or suction procedures, all of which are described below.

Lung region isolation is a treatment for emphysema or other lung ailments that includes the use of devices that isolate a diseased region of the lung in order to modify the air flow to the lung region or to achieve volume reduction or collapse of the lung region. According to the lung region isolation treatment, one or more bronchial isolation devices are implanted in one or more bronchial passageways feeding the targeted region of the lung. The bronchial isolation devices regulate fluid flow through the bronchial passageways. The bronchial isolation devices can be, for example, one-way valves that allow flow in only one direction (e.g., the exhalation direction), occluders or plugs that prevent flow in either direction, or two-way valves that control flow in both directions.

The following references describe exemplary bronchial isolation devices: U.S. Pat. No. 5,954,766 entitled “Body Fluid Flow Control Device”; U.S. Pat. No. 6,694,979, entitled “Methods and Devices for Use in Performing Pulmonary Procedures”; and U.S. patent application Ser. No. 10/270,792, entitled “Bronchial Flow Control Devices and Methods of Use”. The foregoing references are all incorporated by reference in their entirety and are all assigned to Emphasys Medical, Inc., the assignee of the instant application.

The bronchial isolation device can be implanted in a target bronchial passageway using a delivery catheter that is placed through the trachea (via the mouth or the nasal cavities) and into the target location in the bronchial passageway.

Temporary bronchial occlusion procedures involve the occlusion of one or more bronchial passageways in the lung. Such procedures include the use of a catheter having an inflatable balloon that is inserted into the lung. The balloon is positioned in a bronchial passageway and inflated in order to stop bleeding in the lungs (hemoptysis), for example, or for other reasons.

Pursuant to drug delivery, lavage or suction procedures, catheters are placed in the lung to suction fluids, to instill saline for bronchoalveolar lavage, to instill therapeutic compounds, or for other reasons.

For all of the foregoing procedures, a distal region of the catheter must be placed into a bronchial passageway at a specific location in the lungs. Conventional bronchial isolation delivery catheters, as well as commercially-available balloon and suction catheters, incorporate shafts that are straight in a default state. It can be very challenging to position a straight shaft of the catheter into bronchial passageways that are located in certain locations in the lung. Such difficult-to-reach locations include the apical segments of the upper lobes of the lung, as accessing bronchial passageways in such segments requires the delivery catheter to bend through a very extreme angle, sometimes greater than 180 degrees. Often it is difficult, if not impossible, to place the catheters in such locations. Given this problem, there is a need for devices and methods to allow delivery of bronchial isolation device delivery catheters and other bronchial catheters to any location within the bronchial tree of the lungs.

### SUMMARY

Disclosed herein are catheters designs that incorporate shaped or non-straight catheter shafts that facilitate insertion of the catheter shaft into bronchial passageways located in difficult-to-access regions of the lung.

Disclosed is a delivery system for insertion into a lung. The delivery system comprises an elongate endoscope having a proximal end and a distal end and defining an internal lumen, wherein the endoscope is configured to articulate in one or more directions; and a catheter comprising an elongate shaft sized to be inserted through the internal lumen of the endoscope, wherein the catheter has a shape that aids in the articulation of the distal end of the endoscope when the catheter is positioned inside the endoscope.

In another aspect, there is disclosed a catheter for insertion into a lung. The catheter comprises an elongate shaft sized to be inserted into a bronchial tree of the lung, wherein the catheter has at least one bend that permits the catheter to be navigated to a lung region through a bronchial pathway that requires the catheter to articulate across at least one extreme angle.

In another aspect, there is disclosed a catheter for insertion into a lung. The catheter comprises an elongate shaft sized to be inserted into a bronchial passageway of the lung. The shaft is sized to be inserted through the internal lumen of an endoscope configured to articulate in one or more directions. The catheter has a shape that emulates the shape of the endoscope in an articulated state.

The details of one or more embodiments are set forth in the accompanying drawings and the description below. Other features and advantages will be apparent from the description and drawings, and from the claims.

### BRIEF DESCRIPTION OF THE DRAWINGS

FIG. 1 shows an anterior view of a pair of human lungs and a bronchial tree with a bronchial isolation device implanted in a bronchial passageway to bronchially isolate a region of the lung.

FIG. 2 illustrates an anterior view of a pair of human lungs and a bronchial tree.

FIG. 3 illustrates a lateral view of the right lung.

FIG. 4 illustrates a lateral view of the left lung.

FIG. 5 illustrates an anterior view of the trachea and a portion of the bronchial tree.

FIG. 6 shows a perspective view of a bronchoscope.

FIG. 7A shows a distal region of the bronchoscope articulated to an exemplary maximum bend of 180 degrees.

FIG. 7B shows the bronchoscope with a straight-shaft catheter positioned in the working channel and the bronchoscope articulated to its maximum bend.

FIGS. 8A and 8B show the distal regions of two exemplary catheters that have pre-shaped bends along their shafts.

FIG. 9 shows a delivery catheter for delivering a bronchial isolation device to a target location in a body passageway.

FIG. 10 shows a sectioned view of the lung showing an example of a bronchoscope inserted into the bronchial tree of a patient at a difficult-to-reach location.

FIG. 11 shows a sectioned view of the lung showing a catheter having a protruding distal region with a bend.

FIG. 12 shows a portion of a catheter having a distal bend.

### DETAILED DESCRIPTION

Unless defined otherwise, all technical and scientific terms used herein have the same meaning as is commonly understood by one of skill in the art. It should be noted that the various devices and methods disclosed herein are not limited to the treatment of emphysema, and may be used for various other lung diseases.

Disclosed herein are catheters that incorporate shaped or non-straight catheter shafts that facilitate insertion of the catheter shaft into bronchial passageways located in difficult-to-access regions of the lung. Such regions can be characterized, for example, by bronchial passageways that require the catheter to bend at a very extreme angle, sometimes greater than 180 degrees. The apical segments of the upper lobes of the lung are examples of difficult-to-access regions.

The catheters are described herein in the context of delivery catheters that are used to deliver bronchial isolation devices (which are sometimes referred to herein as flow control devices) into the lung. However, it should be appreciated that the pre-shaped catheters described herein can be various types of catheters for use in the lung, including suction catheters, drug delivery catheters, stent delivery catheters, or any other type of catheter and not just the bronchial isolation delivery catheter shown here as an example. It should also be appreciated that although a bronchoscope is used in the exemplary embodiments described below, the devices and methods described herein are suitable for use with any flexible endoscope.

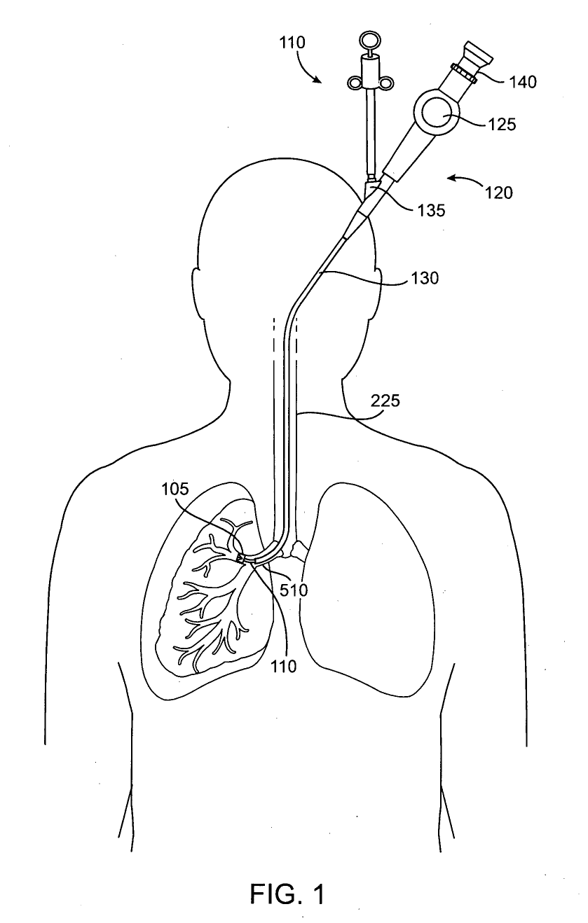

FIG. 1 shows an exemplary embodiment wherein a bronchial isolation device 105 is deployed into a bronchial passageway using a delivery catheter 110. The delivery catheter 110 has been inserted into the bronchial passageway through the patient's trachea 225 such that a distal region of the delivery catheter 110 is positioned at a target location in the bronchial passageway. Prior to inserting the delivery catheter 110 into the lung, the bronchial isolation device 105 is removably mounted to a distal region of the delivery catheter 110. Once the distal region of the delivery catheter 110 is positioned at the target location in the bronchial passageway, the bronchial isolation device is ejected from the housing and deployed within the passageway.

The delivery catheter 110 may be guided to the target location in the bronchial passageway pursuant to various methods. According to one method, the delivery catheter 110 has a central lumen, and the delivery catheter 110 is guided to the target location by sliding the delivery catheter 110 over a guidewire that has previously been passed through the trachea to the target location in the bronchial passageway.

In another method, shown in FIG. 1, a bronchoscope 120 assists in the insertion of the delivery catheter 110 through the trachea and into the bronchial passageway. The method that uses the bronchoscope 120 is referred to as the “transcopic” method. According to the transcopic method, the delivery catheter 110 is inserted into the working channel of the bronchoscope 120. The bronchoscope 120 is inserted through the trachea and into the bronchial tree of the patient. The bronchoscope and catheter pass along a bronchial pathway that can require the bronchoscope and catheter to bend at extreme angles. Extreme angles can be characterized, for example, by being greater than ninety degrees, greater than 120 degrees, greater than 150 degrees, or greater than 180 degrees. Once the bronchoscope is positioned properly, the distal tip of the bronchoscope 120 is placed just proximally to the ostium of the target bronchial passageway. The distal region of the delivery catheter 110, which is positioned inside the working channel of the bronchoscope, thus has access to the target bronchial passageway. The delivery catheter 110 can be inserted into the working channel of the bronchoscope either before or after the bronchoscope 120 is inserted into the lung.

The following references describe exemplary bronchial isolation devices and delivery devices: U.S. Pat. No. 5,954,766 entitled “Body Fluid Flow Control Device”; U.S. patent application Ser. No. 09/797,910, entitled “Methods and Devices for Use in Performing Pulmonary Procedures”; U.S. patent application Ser. No. 10/270,792, entitled “Bronchial Flow Control Devices and Methods of Use”; and U.S. patent application Ser. No. 10/448,154, entitled “Guidewire Delivery of Implantable Bronchial Isolation Devices in Accordance with Lung Treatment”. The foregoing references are all incorporated by reference in their entirety and are all assigned to Emphasys Medical, Inc., the assignee of the instant application.

Conventional catheters have shafts that are straight in a default state. It has been observed that it can be difficult to insert such straight-shaft catheters into certain regions of the lung, as the straight-shaft catheters resist bending to the degree that is required to access such regions. Disclosed herein are various catheter designs that incorporate shaped or non-straight catheter shafts to aid in the placement of bronchial isolation devices (or other bronchially implanted devices) into bronchial passageways that are difficult to access.

Exemplary Lung Regions

Throughout this disclosure, reference is made to the term “lung region”. As used herein, the term “lung region” refers to a defined division or portion of a lung. For purposes of example, lung regions are described herein with reference to human lungs, wherein some exemplary lung regions include lung lobes and lung segments. Thus, the term “lung region” as used herein can refer, for example, to a lung lobe or a lung segment. Such nomenclature conform to nomenclature for portions of the lungs that are known to those skilled in the art. However, it should be appreciated that the term “lung region” does not necessarily refer to a lung lobe or a lung segment, but can refer to some other defined division or portion of a human or non-human lung.

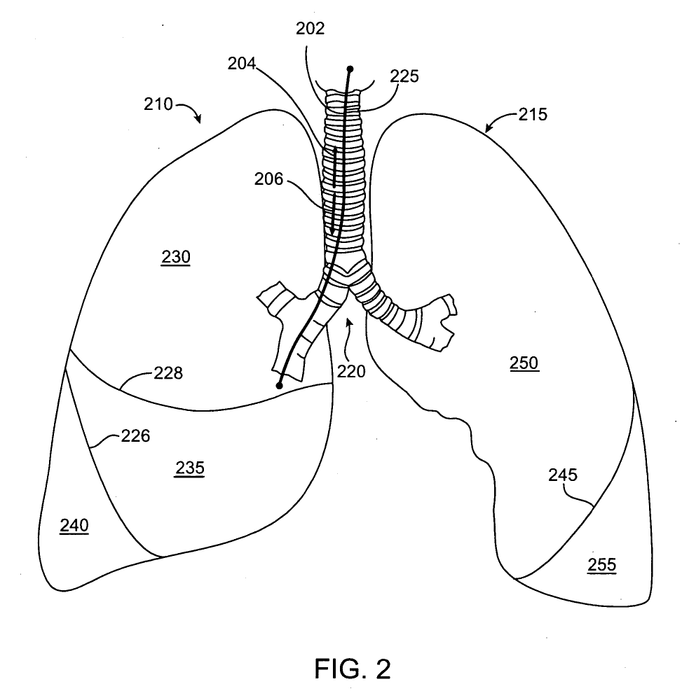

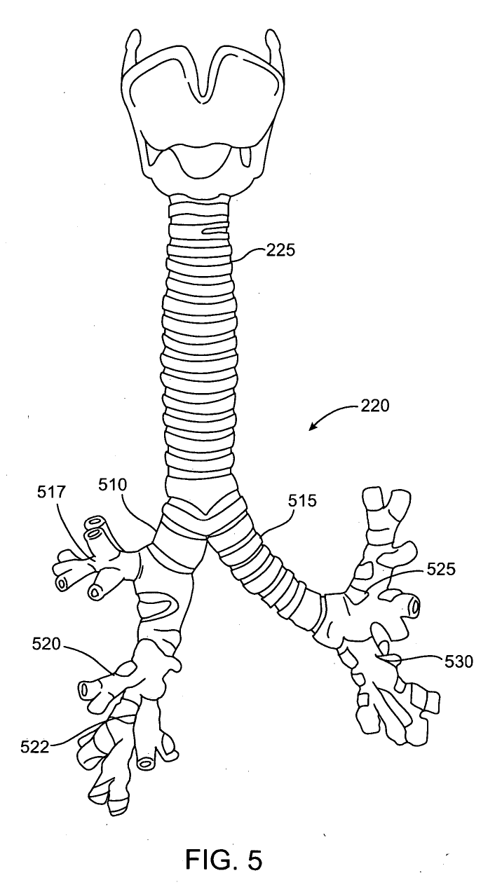

FIG. 2 shows an anterior view of a pair of human lungs 210, 215 and a bronchial tree 220 that provides a fluid pathway into and out of the lungs 210, 215 from a trachea 225, as will be known to those skilled in the art. As used herein, the term “fluid” can refer to a gas, a liquid, or a combination of gas(es) and liquid(s). For clarity of illustration, FIG. 2 shows only a portion of the bronchial tree 220, which is described in more detail below with reference to FIG. 5.

Throughout this description, certain terms are used that refer to relative directions or locations along a path defined from an entryway into the patient's body (e.g., the mouth or nose) to the patient's lungs. The path of airflow into the lungs generally begins at the patient's mouth or nose, travels through the trachea into one or more bronchial passageways, and terminates at some point in the patient's lungs. For example, FIG. 2 shows a path 202 that travels through the trachea 225 and through a bronchial passageway into a location in the right lung 210. The term “proximal direction” refers to the direction along such a path 202 that points toward the patient's mouth or nose and away from the patient's lungs. In other words, the proximal direction is generally the same as the expiration direction when the patient breathes. The arrow 204 in FIG. 2 points in the proximal or expiratory direction. The term “distal direction” refers to the direction along such a path 202 that points toward the patient's lung and away from the mouth or nose. The distal direction is generally the same as the inhalation or inspiratory direction when the patient breathes. The arrow 206 in FIG. 2 points in the distal or inhalation direction.

The lungs include a right lung 210 and a left lung 215. The right lung 210 includes lung regions comprised of three lobes, including a right upper lobe 230, a right middle lobe 235, and a right lower lobe 240. The lobes 230, 235, 240 are separated by two interlobar fissures, including a right oblique fissure 226 and a right transverse fissure 228. The right oblique fissure 226 separates the right lower lobe 240 from the right upper lobe 230 and from the right middle lobe 235. The right transverse fissure 228 separates the right upper lobe 230 from the right middle lobe 235.

As shown in FIG. 2, the left lung 215 includes lung regions comprised of two lobes, including the left upper lobe 250 and the left lower lobe 255. An interlobar fissure comprised of a left oblique fissure 245 of the left lung 215 separates the left upper lobe 250 from the left lower lobe 255. The lobes 230, 235, 240, 250, 255 are directly supplied air via respective lobar bronchi, as described in detail below.

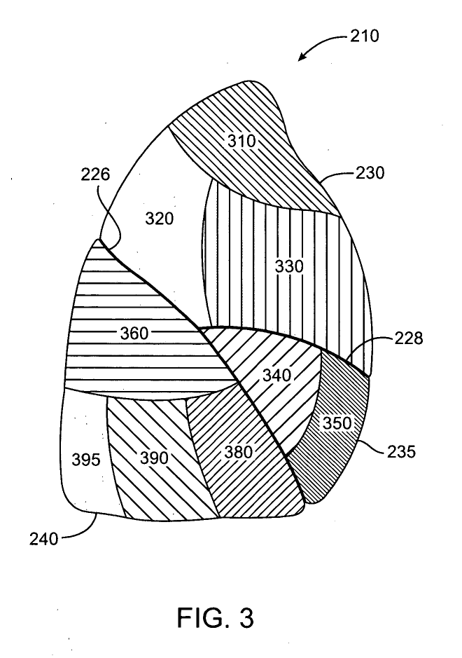

FIG. 3 is a lateral view of the right lung 210. The right lung 210 is subdivided into lung regions comprised of a plurality of bronchopulmonary segments. Each bronchopulmonary segment is directly supplied air by a corresponding segmental tertiary bronchus, as described below. The bronchopulmonary segments of the right lung 210 include a right apical segment 310, a right posterior segment 320, and a right anterior segment 330, all of which are disposed in the right upper lobe 230. The right lung bronchopulmonary segments further include a right lateral segment 340 and a right medial segment 350, which are disposed in the right middle lobe 235. The right lower lobe 240 includes bronchopulmonary segments comprised of a right superior segment 360, a right medial basal segment (which cannot be seen from the lateral view and is not shown in FIG. 3), a right anterior basal segment 380, a right lateral basal segment 390, and a right posterior basal segment 395.

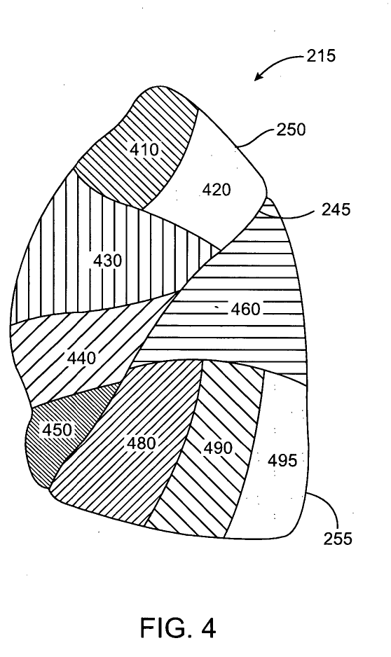

FIG. 4 shows a lateral view of the left lung 215, which is subdivided into lung regions comprised of a plurality of bronchopulmonary segments. The bronchopulmonary segments include a left apical segment 410, a left posterior segment 420, a left anterior segment 430, a left superior segment 440, and a left inferior segment 450, which are disposed in the left lung upper lobe 250. The lower lobe 255 of the left lung 215 includes bronchopulmonary segments comprised of a left superior segment 460, a left medial basal segment (which cannot be seen from the lateral view and is not shown in FIG. 4), a left anterior basal segment 480, a left lateral basal segment 490, and a left posterior basal segment 495.

FIG. 5 shows an anterior view of the trachea 325 and a portion of the bronchial tree 220, which includes a network of bronchial passageways, as described below. The trachea 225 divides at a lower end into two bronchial passageways comprised of primary bronchi, including a right primary bronchus 510 that provides direct air flow to the right lung 210, and a left primary bronchus 515 that provides direct air flow to the left lung 215. Each primary bronchus 510, 515 divides into a next generation of bronchial passageways comprised of a plurality of lobar bronchi. The right primary bronchus 510 divides into a right upper lobar bronchus 517, a right middle lobar bronchus 520, and a right lower lobar bronchus 422. The left primary bronchus 415 divides into a left upper lobar bronchus 525 and a left lower lobar bronchus 530. Each lobar bronchus 517, 520, 522, 525, 530 directly feeds fluid to a respective lung lobe, as indicated by the respective names of the lobar bronchi. The lobar bronchi each divide into yet another generation of bronchial passageways comprised of segmental bronchi, which provide air flow to the bronchopulmonary segments discussed above.

As is known to those skilled in the art, a bronchial passageway defines an internal lumen through which fluid can flow to and from a lung or lung region. The diameter of the internal lumen for a specific bronchial passageway can vary based on the bronchial passageway's location in the bronchial tree (such as whether the bronchial passageway is a lobar bronchus or a segmental bronchus) and can also vary from patient to patient. However, the internal diameter of a bronchial passageway is generally in the range of 3 millimeters (mm) to 10 mm, although the internal diameter of a bronchial passageway can be outside of this range. For example, a bronchial passageway can have an internal diameter of well below 1 mm at locations deep within the lung. The internal diameter can also vary from inhalation to exhalation as the diameter increases during inhalation as the lungs expand, and decreases during exhalation as the lungs contract.

Catheter Shaped to Assist in Flexible Endoscope Articulation

Flexible endoscopes are routinely used to access, view and perform procedures on various internal parts of the body. The distal region of the flexible endoscope can be designed to articulate in one or more directions in order to allow the tip to be steered and advanced into the body cavity being examined. For example, the tip of a bronchoscope used to examine the bronchial tree of the lungs can articulate or bend 180 degrees in one direction, and 130 degrees in the opposite direction.

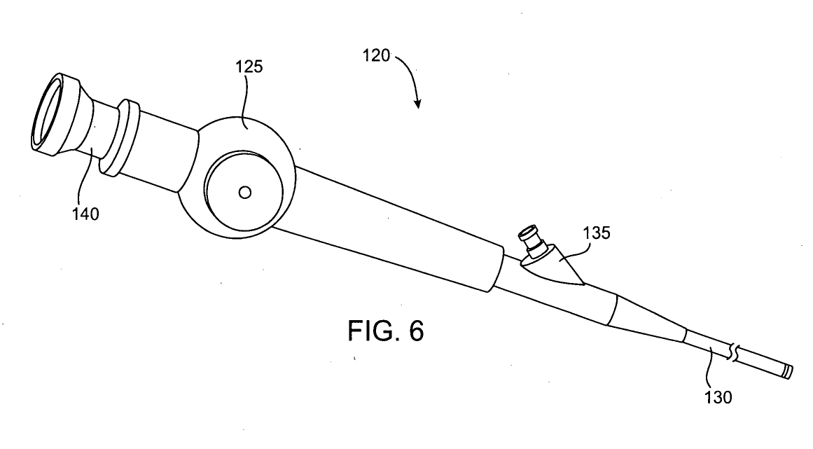

FIG. 6 shows an enlarged view of a bronchoscope 120, including a steering mechanism 125, delivery shaft 130, working channel entry port 135, and visualization eyepiece 140. A working channel (sometimes referred to as a biopsy channel) extends through the delivery shaft 130 and communicates with the entry port 135 at the proximal end of the bronchoscope 120. The bronchoscope 120 can also include various other channels, such as a visualization channel that communicates with the eyepiece 140 and one or more illumination channels. It should be appreciated that the bronchoscope can have a variety of configurations and is not limited to the embodiment shown in the figures.

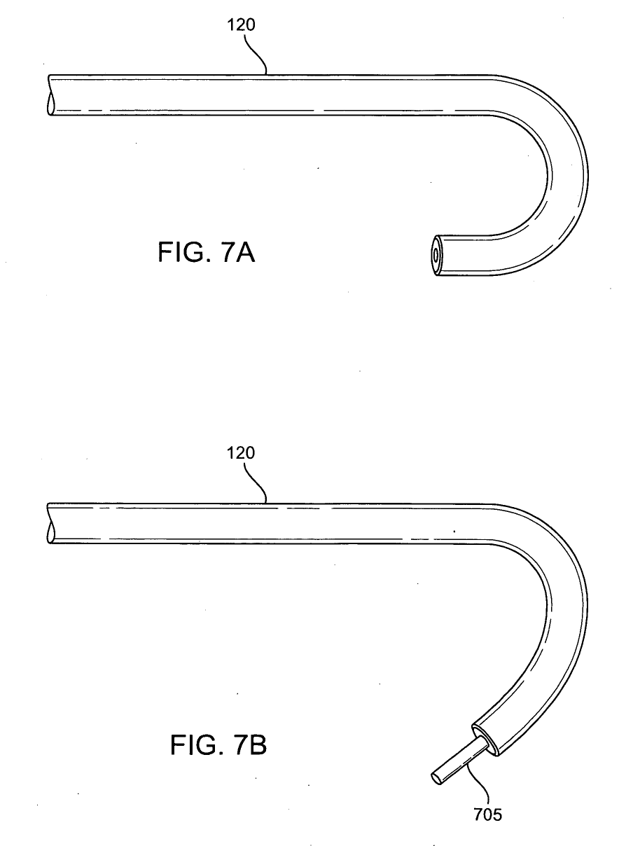

As mentioned, the distal region of the shaft 130 is configured to articulate in one or more directions in order to allow the distal tip to be steered and advanced into the bronchial passageways in the lung. For example, FIG. 7A shows the distal region of the bronchoscope 120 articulated to an exemplary maximum bend of 180 degrees relative to a longitudinal axis of the shaft when straightened. It should be appreciated that the bronchoscope can be designed to have maximum bends that vary in degree and that the maximum bend need not be 180 degrees.

During a lung interventional procedures (such as a bronchial isolation procedure), a catheter is positioned inside the working channel of the bronchoscope 120. The presence of the catheter inside the working channel can decrease the maximum articulation for the bronchoscope shaft. This is because all catheters used in the body have some bending stiffness or resistance to bending, which increases the resistance to bending of the bronchoscope. That is, when a catheter is positioned in the working channel of a flexible bronchoscope, the distal region of the catheter must bend along with the bronchoscope when the distal region of the bronchoscope is articulated. The inherent resistance of the catheter to bending results in the maximum articulation of the tip of the bronchoscope being reduced from that which could otherwise be achieved without a catheter in the working channel.

This is described in more detail with reference to an example in FIG. 7B, which shows the bronchoscope 120 with a straight catheter 705 positioned in the working channel and the bronchoscope 120 articulated to its maximum bend. The catheter 705, which is straight in a default state, is shown protruding outwardly from the distal end of the bronchoscope 120. The maximum angle of articulation of the distal region of the bronchoscope 120 is reduced to approximately 120 degrees due to the presence of the straight catheter 705 in the working channel (versus the maximum angle of articulation of 180 degrees when the straight catheter 705 is not present, as shown in FIG. 7A.) Thus, the presence of the straight catheter 705 in the working channel effectively reduces the maximum angle of articulation or bend of the bronchoscope 120.

This reduction in the angle of articulation of the distal region of the bronchoscope can make it very difficult or impossible to transcopically place a catheter in a bronchial passageway positioned at a location that requires a high degree of articulation of the bronchoscope to be accessed. In the human lung, the apical segment of the right upper lobe and the apicoposterior segment of the left upper lobe both require nearly a full 180 degree articulation (relative to a longitudinal axis of the bronchoscope when straightened) of the distal tip of the bronchoscope in order to be accessed by the bronchoscope. When a straight-shaft catheter is inserted into the working channel of the bronchoscope, it can become difficult or impossible to place the catheter into a target bronchial passageway located in a difficult-to-access region, such as, for example, the apical segment of the right upper lobe or the apicoposterior segment of the left upper lobe.

There is now described a catheter 110 that mitigates or eliminates the reduction in maximum articulation of a bronchoscope having a catheter positioned in its working channel. The catheter 110 has a shaft that is non-straight in a default state. That is, the catheter 110 has a shaft that is bent or that has some predetermined curvature along a region of the shaft that renders the shaft non-straight in a default state. In one embodiment, the shaft of the catheter 110, rather than being straight, has a shape that emulates the shape of region of a maximally articulated bronchoscope. For example, the catheter shaft can have a shape that emulates the bent shape of the distal of the bronchoscope 120 shown in FIG. 7A.

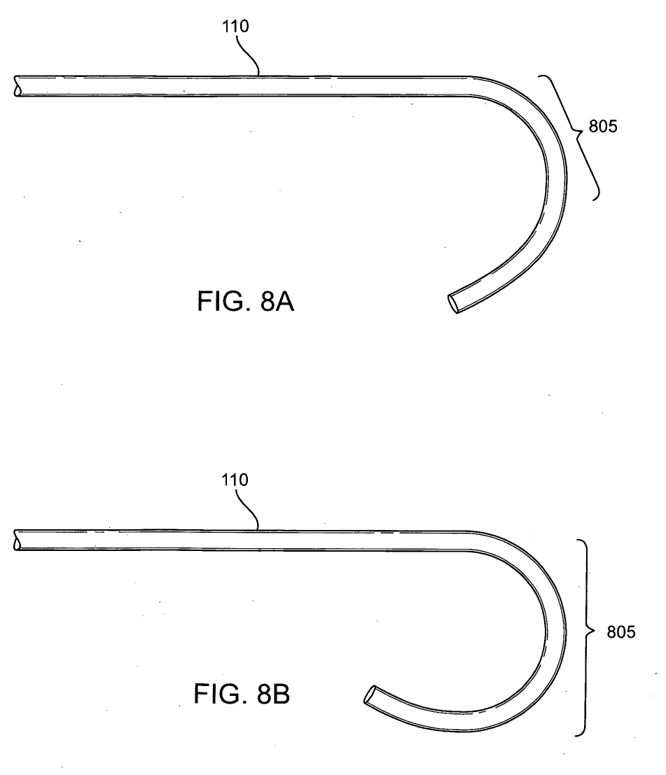

FIGS. 8A and 8B show the distal regions of two exemplary catheters 110 that have pre-shaped bends 805 along the shafts. The bends 805 in the catheters 110 are present in a default state when no net force is being exerted on the catheter shaft. If a sufficient force is applied to the catheter shafts, the bends 805 can be straightened, although the catheter shafts return to the default, bent shape when such force is removed.

The configuration of the bends 805 can vary. In one embodiment, the bend emulates a bend in a corresponding bronchoscope with which the catheter 110 is used. The bend in the bronchoscope can be a bend that is present in a default state such that the bronchoscope is non-straight in the default state. In another embodiment, the bend 805 is selected to conform to or approximate the shape of a corresponding bronchoscope that has been articulated to its maximum bend, such as the bronchoscope shown in FIG. 7A. Thus, the bend 805 can emulate a bend in a bronchoscope that has a bend in a default state, or the bend 805 can conform to or approximate the bent shape of a bronchoscope that has been articulated to a maximum bend (whether or not the bronchoscope has a pre-shaped bend.)

The greater the angle of the pre-shaped bend in the catheter 110, the greater the resultant maximal articulation of the bronchoscope or endoscope in which the catheter is inserted. The catheter 110 shown in FIG. 8A has a pre-shaped bend 805 that is less than 180 degrees (relative to a longitudinal axis of the catheter shaft when the catheter is straightened). It has been observed that if the catheter 110 having the pre-shaped bend of FIG. 8A is inserted into the bronchoscope, the maximum articulation of the bronchoscope is reduced by approximately 10 degrees from the articulation achieved without a catheter in the working channel (as shown in FIG. 7A.) The maximum articulation of the bronchoscope is reduced by an even greater amount when a straight-shaft catheter is present in the working channel.

The catheter 110 shown in FIG. 8B has an even greater degree of pre-shaped bend 805 than that shown in FIG. 8A. When a catheter having the shape shown in FIG. 8A is used in the bronchoscope, the reduction in maximum articulation of the bronchoscope can be completely eliminated. However, at a relatively extreme bends (such as greater than 180 degrees) the catheter 110 can become difficult to handle and can be difficult to insert into the working channel entry port 135 of the bronchoscope. As mentioned, the pre-shaped catheter is not limited to use with a bronchoscope and are suitable for use with any flexible endoscope.

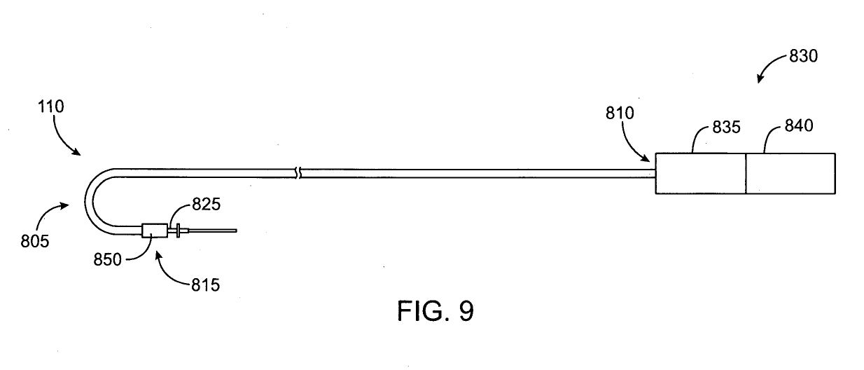

The pre-shaped bend configuration of the catheter shaft can be incorporated into various types of catheters. In one embodiment, shown in FIG. 9, the catheter 110 comprises a delivery catheter 110 for delivering and deploying the bronchial isolation device 105 to a target location in a bronchial passageway. The delivery catheter 110 has a proximal end 810 and a distal end 815 that can be deployed to a target location in a patient's bronchial passageway, such as through the trachea. A pre-shaped bend 805 is located at a distal region of the catheter 110. The amount of bend in the pre-shaped bend 805 can vary or can be selected to conform to the maximum bend of a corresponding bronchoscope. It should be appreciated that the catheter 110 is not shown to scale in FIG. 9.

The catheter 110 has an elongated outer shaft 820 and an elongated inner shaft 825 that is slidably positioned within the outer shaft 820 such that the outer shaft 820 can slidably move relative to the inner shaft 825 along the length of the catheter. The outer shaft 820 and inner shaft 825 can be manufactured of various materials. In one embodiment, the outer shaft 820 is manufactured of a composite stainless steel wire reinforced polymer and the inner shaft 825 is manufactured of solid nitinol.

With reference still to FIG. 9, an actuation handle 830 is located at the proximal end 810 of the catheter 110. The actuation handle 830 can be actuated to slidably achieve relative axial movement between the outer shaft 820 and the inner shaft 825. During such movement, the outer shaft 820 and inner shaft 825 slide relative to one another. Generally, the handle 830 includes a first piece 835 and a second actuation piece 840, which is moveable relative to the first piece 835. The outer shaft 820 of the catheter 110 can be moved relative to the inner shaft 825 by moving the first piece 835 of the handle 830 relative to the second piece 840. Other means of actuation, such as means that do not require a first piece and a second piece, can also be used.

With reference still to FIG. 9, a housing 850 is located at or near a distal end of the catheter 110 for holding therein the bronchial isolation device 105. The housing 850 defines an inner cavity that is sized to receive the bronchial isolation device 115 in a compressed state. The bronchial isolation device 105 can be ejected from the housing by actuating the handle 830 to cause the inner shaft 825 to slide relative to the outer shaft 820 (or vice-versa). The inner shaft has a pushing element mounted on the distal end, and this bears against the compressed bronchial isolation device 805 and pushes it out of the housing as the inner shaft 825 and outer shaft 825 slide relative to one another.

As mentioned, in one embodiment, the outer shaft 820 is manufactured of a composite stainless steel wire reinforced polymer and the inner shaft 825 is manufactured of solid nitinol. The bending stiffness of the catheter 110 can be determined by the bending stiffness of the nitinol inner shaft 825. In this regard, the pre-shaped configuration of the catheter 110 can be achieved by heat treating the nitinol inner shaft 825 in the desired curved shape while leaving the outer shaft 820 straight. Thus, the inner shaft 825 can have a pre-shaped bend in a default state while the outer shaft is straight in a default state. The resultant catheter 110 has a pre-shaped bend that is determined by the bend of the inner shaft 825. Alternately, the outer shaft 820 can have a pre-shaped bend and the inner shaft 825 can be straight in a default state. The combination of the pre-shaped outer shaft and straight inner shaft results in a catheter 110 having a pre-shaped bend.

The amount of maximum bend and the amount of default bend of the catheter depends on the shape of the bent portion (in either the inner shaft, outer shaft, or both) and by the relative stiffness between the materials of the inner and outer shafts. For example, the inner shaft can have a first bending stiffness and the outer shaft can have a second bending stiffness that is different than or the same than the first bending stiffness. If the bending stiffness of the inner shaft is greater than the bending stiffness of the outer shaft (i.e., the inner shaft is more resistant to bending than the outer shaft), the final shape of the bend catheter 110 is most likely dictated by the shape of the inner shaft. The final degree of the bend of catheter 110 is of a degree that is somewhere between the bend in the outer shaft and the bend in the inner shaft or equal to the bend in both shafts if the bends are the same. The relative stiffness and shapes of the bends for inner and outer shafts can be selected to provide a desired bend shape and maximum articulation for the catheter 110.

Catheter with Shaped Distal End

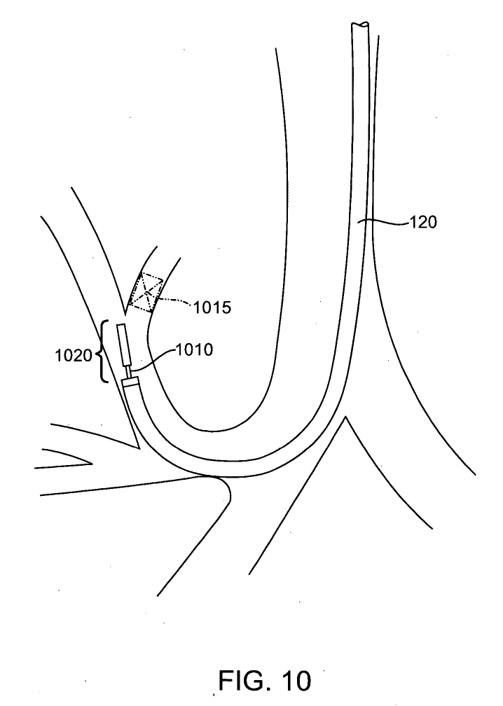

Situations can arise wherein the articulation of the end of the endoscope is insufficient to allow a standard straight-shaft catheter that has been threaded through the working channel of the endoscope to be placed into difficult to access or tortuous anatomy. FIG. 10 shows a sectioned view of the lung showing an example of a bronchoscope 120 inserted into the bronchial tree of a patient at a difficult-to-reach location.

The bronchoscope 120 has been maximally articulated, although the maximum articulation achieved by the bronchoscope 120 is insufficient to allow a catheter 1010 inserted through the working channel to access the target location 1015 of the bronchial passageway. In the example shown in FIG. 10, target location 1015 is located in an apical sub-segment of the right apical lobe of the human lungs. A distal-most region 1020 of the catheter 1010 protrudes outwardly of the distal tip of the bronchoscope 120. The tip of the region 1020 cannot access the target location 1015 due to the insufficient maximum articulation of the bronchoscope 120.

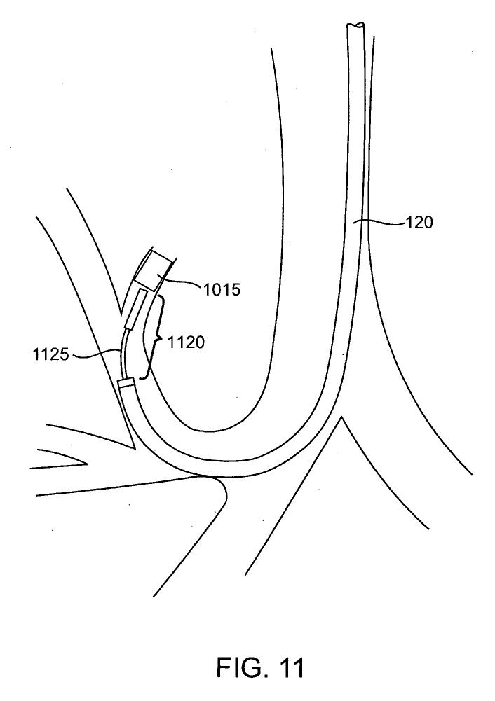

One solution to the access problem shown in FIG. 10 is to configure the catheter 1005 such that the region that protrudes out of distal end of the bronchoscope 120 is bent in one or more directions. FIG. 11 shows a sectioned view of the lung showing a catheter 1110 having a protruding distal region 1120 with a bend 1125. Thus, the distal region 1120 of the catheter 1110 bends in one direction after it is pushed out of the distal tip of the bronchoscope 120. If the catheter 1110 is then rotated relative to the bronchoscope 120, the direction of the deflection of the tip of the catheter 1110 may be adjusted to align with the target location 1015.

When the catheter 1110 inserted into the working channel of the bronchoscope 120, the bend 1125 in the distal region 1120 straightens or otherwise adjusts to conform to the shape of the bronchoscope 120. When the distal region 1120 of the catheter is pushed out of the distal end of the bronchoscope 120, the bend 1125 assumes its default, bent shape. The bend 1125 cause the region 1120 of the catheter 1110 to deflect away from the direction of the axial centerline of the working channel of the bronchoscope 120. The catheter can then be rotated to alter the deflection direction of the catheter to align the catheter with the target location 1015. Thus, the target location 1015 that was previously difficult to access (as shown in FIG. 9) can be reached due to the bend 1125 in the region 1120 and the catheter can be successfully inserted.

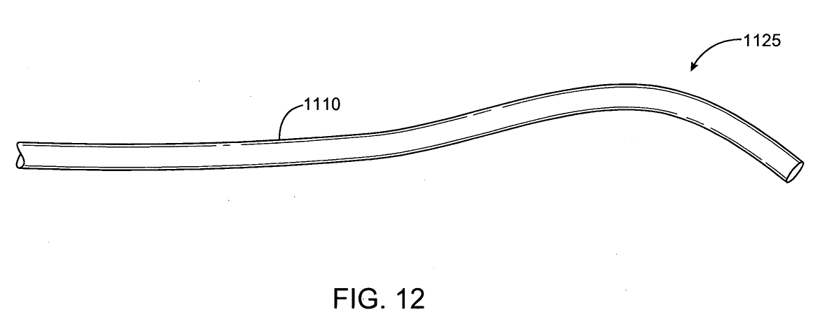

FIG. 12 shows a portion of the catheter 1110 having the bend 1125. The bend 1125 in the catheter 1110 is desirably as close to the distal tip of the catheter as possible, unlike in the catheter 110 (shown in FIGS. 8A, 8B, and 9) where the bend 805 is located somewhat proximally to the distal tip so as to coincide with the articulation bend in the endoscope. In one embodiment, the bend 1125 is located in the distal region 1120 of the catheter 1110 that protrudes outwardly of the distal end of the bronchoscope 120 during use of the bronchoscope and catheter in the lung. In an exemplary implementation, the distal region 1120 has a length in the range of 50 mm to 160 mm from the distal tip of the catheter 1110, though the length could be shorter or longer than this range. It should be appreciated that the length of the distal region 1120 that protrudes outwardly from the bronchoscope can vary based on the anatomy of the patient, on the location in the bronchial tree where the procedure is performed, or for other reasons.

Moreover, the bend 1125 is not required to be located in the region 1120 of the catheter that protrudes outwardly from the bronchoscope. The bend 1125 can be located within a portion of the catheter 1110 that remains inside the working channel as long as the bend has a location that permits the distal tip of the catheter to deflect away from the direction of the axial centerline of the working channel of the bronchoscope.

It should be appreciated that a catheter can have a bend 1125 (of the type described with reference to FIGS. 10-12) in combination with a bend 805 that emulates a bend in the bronchoscope (as shown in FIGS. 8A, 8B, and 9.) Alternately, the catheter can have only the bend 1125 without the bend 805 that emulates a bronchoscope bend. It should further be appreciated that though the catheters sometimes described herein are bronchial isolation device delivery catheters, other catheters such as suction catheters, drug delivery catheters, stent delivery catheters, or any other type of catheter can incorporate the bend configurations. It should also be appreciated that a bronchoscope was used in the exemplary embodiments described above; however the devices and methods described are suitable for use with any flexible endoscope.

Although embodiments of various methods and devices are described herein in detail with reference to certain versions, it should be appreciated that other versions, embodiments, methods of use, and combinations thereof are also possible. Therefore the spirit and scope of the appended claims should not be limited to the description of the embodiments contained herein.

What is claimed:

1. A delivery system for insertion into a lung, comprising:
an elongate endoscope having a proximal end and a distal end and defining an internal lumen, the endoscope configured to articulate in one or more directions; and
a catheter comprising an elongate shaft sized to be inserted through the internal lumen of the endoscope, wherein the catheter has a shape that aids in the articulation of the distal end of the endoscope when the catheter is positioned inside the endoscope.

2. A system as in claim 1, wherein the catheter has a shape that is selected to complement articulation of the distal end of the bronchoscope.

3. A system in claim 1, wherein the catheter has a first bend shaped to complement a shape of a bend in the endoscope.

4. A delivery system as in claim 1, wherein the catheter is configured to deliver a flow control device into a lung, the flow control device being removably mounted on a housing at a distal end of the catheter.

5. A system as in claim 1, wherein the catheter comprises an outer shaft and an inner shaft, the inner shaft having a first shape and a first stiffness and the outer shaft having a second shape and a second stiffness, wherein the first shape, first stiffness, second shape, and second stiffness have relative values that determine the shape of the catheter.

6. A system as in claim 5, wherein the first stiffness differs from the second stiffness.

7. A system as in claim 5, wherein the first shape and second shape both include a bend, and wherein the bends are different.

8. A system as in claim 1, wherein the catheter includes a bend on a distal end of the catheter.

9. A system as in claim 1, wherein the catheter has a first bend shaped to complement a shape of a bend in the endoscope and a second bend near a distal end of the catheter, wherein the second bend is located nearer the distal end of the catheter than the first bend.

10. A system as in claim 9, wherein the second bend causes a distal end of the catheter to deflect away from a centerline of the internal lumen in the endoscope.

11. A system as in claim 1, wherein the endoscope comprises a bronchoscope

12. A catheter for insertion into a lung, comprising
an elongate shaft sized to be inserted into a bronchial tree of the lung, wherein the catheter has at least one bend that permits the catheter to be navigated to a lung region through a bronchial pathway that requires the catheter to articulate across at least one extreme angle.

13. A catheter as in claim 12, wherein the extreme angle is greater than 90 degrees.

14. A catheter as in claim 12, wherein the extreme angle is greater than 120 degrees.

15. A catheter as in claim 12, wherein the extreme angle is greater than 150 degrees.

16. A catheter as in claim 12, wherein the extreme angle is greater than 180 degrees.

17. A catheter as in claim 12, wherein the at least one bend comprises a single bend that emulates a bend in a corresponding bronchoscope.

18. A catheter as in claim 12, wherein the at least one bend comprises a single bend that is shaped to aid in the articulation of a distal end of a bronchoscope.

19. A catheter as in claim 12, wherein the at least one bend comprises a single bend that is located within a distal region of the catheter, wherein the distal region of the catheter is located within 50 millimeters to 160 millimeters from a distal end of the catheter.

20. A catheter as in claim 12, wherein the at least one bend in the catheter includes a first bend that is shaped to aid in the articulation of a distal end of a bronchoscope and a second bend that is located distally of a distal end of the bronchoscope when the catheter is positioned in a working channel of the bronchoscope.

21. A catheter as in claim 12, wherein the lung region is an apical segment of an upper lobe of the lung.

22. A catheter for insertion into a lung, comprising:
an elongate shaft sized to be inserted into a bronchial passageway of the lung, the shaft sized to be inserted through an internal lumen of an endoscope configured to articulate in one or more directions, wherein the catheter has a shape that emulates the shape of the endoscope in an articulated state.
